(19) United States
(12) Patent Application Publication
(10) Pub. No.: US 2023/0060089 A1
(43) Pub. Date: Feb. 23, 2023

(54) Laser speckle imaging for live cell quantification

(71) Applicant: Nirrin Technologies, Inc., Billerica (MA)

(72) Inventors: Bryan A. Hassell, Cambridge, MA (US); Gregory A. Phelps, Cambridge, MA (US); David P. Marchessault, Hopkinton, MA (US)

(21) Appl. No.: 17/893,279

(22) Filed: Aug. 23, 2022

Related U.S. Application Data
(60) Provisional application No. 63/235,909, filed on Aug. 23, 2021.

(51) Int. Cl. G01N 15/14; C12M 1/34

(52) U.S. Cl. CPC G01N 15/1434; C12M 41/46; G01N 15/1463; G01N 2015/1006

(57) ABSTRACT

A method and system for studying cell viability and protein aggregation. In one aspect, the method relies on speckle information to analyze cells or other structures present in a fluid. A probe for measuring in situ structures includes a tip section having a sample detection region and a camera provided with image sensors.

### RELATED APPLICATIONS

This application claims the benefit under 35 USC 119(e) of U.S. Provisional Application No. 63/235,909, filed on Aug. 23, 2021, which is incorporated herein by reference in its entirety.

### BACKGROUND OF THE INVENTION

Many processes in the chemical, biochemical, pharmaceutical, food, beverage and in other industries require some type of monitoring. Sensors have been developed and are available to measure pH, dissolved oxygen (DO), temperature or pressure in-situ and in real-time. Common techniques for detecting chemical constituents include high performance liquid chromatography (HPLC), gas chromatography-mass spectroscopy (GCMS), or enzyme-and reagent-based electrochemical methods.

Various optical spectroscopy approaches are also available to assess components, also referred to as analytes, in a sample. Among these, probably the most common is absorption spectroscopy. Incident light excites electrons of the analyte from a low energy ground state into a high energy, excited state, and the energy can be absorbed by both non-bonding n-electrons and π-electrons within a molecular orbital. Absorption spectroscopy can be performed in the ultraviolet, visible, and/or infrared region, with analytes of varying material phases and composition being interrogated by specific wavelengths or wavelength bands of light. The resulting transmitted light is then used to resolve the absorbed spectra, to determine the analyte's or sample's composition, temperature, pH and/or other intrinsic properties for applications ranging from medical diagnostics, pharmaceutical development, food and beverage quality control, to list a few.

Another option is Raman spectroscopy, which works by the detection of inelastic scattering of typically monochromatic light from a laser.

Many of the processes being monitored also involve growing and/or maintaining cells in bioreactors along with the harvesting of cells from such bioreactors. In such situations, the viability of the cells cannot be overemphasized.

### SUMMARY OF THE INVENTION

Some of the existing techniques for measuring cell densities (total and/or viable) utilize an exclusion dye, which can be fluorescent. Also known are spectrophotometric methods.

Typical approaches can be destructive as they rely on removing and analyzing samples off-line. This may be particularly problematic when measuring cell densities.

In addition, protocols followed in analyzing cell cultures often require expensive consumables and/or a long time to complete. In many cases, the equipment needed to perform these analyses is expensive, involving calibrations, and trained operators. Procedures may be time- and labor-intensive, problems that are often mitigated by decreasing the sampling frequency of a given process, thus reducing the data points. In many instances, samples are run in batches, after the process has been completed, yielding little or no feedback for adjusting conditions on an ongoing basis. Drawbacks such as these can persist even with automated sampling operations.

A need exists for robust, hands-free, non-destructive, real time techniques for identifying and/or quantifying cell densities and, possibly, other constituents in an ongoing process. For procedures in which cells are used to produce antibodies or procedures in which cells themselves are the product, such as those involved in cell therapy procedures, non-destructive, real time techniques for measuring cell viability are also highly desirable. Also desirable are approaches for monitoring cell viability in situ or in a flow cell, without the need to withdraw samples from the culture vessel being employed, often a bioreactor.

In general, the invention relates to monitoring and assessing structures (cells, proteins and so forth) in samples. In one case, this entails measuring or monitoring cell density and/or viability in ways that address at least some of the problems described above. The invention can also relate to approaches for measuring protein aggregates.

Approaches described herein also can be implemented on a fluid that, in some embodiments, is in a state of flow. In one illustration, a cell culture or protein aggregates (soluble protein aggregates, for instance), optionally in conjunction with monomeric protein levels, are monitored in a loop from a bioreactor or a suitable point during downstream bioprocessing (processing that typically occurs after the separation of the protein-containing fluid from the protein-producing cells). In one implementation, measuring protein aggregation is conducted post protein A column.

Some of the arrangements and/or techniques described herein are configured to probe a bioreactor in situ. It is possible to monitor not only viable cells and proteins, but also other analytes present in the bioreactor's medium.

Aspects of the invention can be practiced in bioprocesses such as those used in the production of monoclonal antibodies or in cell therapy applications, where the cells themselves represent the product to be administered to a subject. Embodiments described herein also can find applications in detecting bioburden in various fields (e.g., various processes in biotechnology or the pharmaceutical industry, wastewater treatment, food and beverage, and so forth).

The advantages of measuring cell viability in situ cannot be overemphasized. Techniques described herein can provide on-line, real time measurements, obtained in a non-destructive manner. Whereas many existing approaches rely on removing and/or circulating cells in loops external to the process vessel, typically through a pumping system, some of the embodiments described herein can reduce, minimize and often eliminate the exposure of cells to conditions external to the bioreactor. In addition, cells are prevented from being drawn into the pumping system.

The invention can find applications in optimizing cell growth, in support of product purification, quality control, product recovery or other stages in the manufacture of proteins or other areas. Techniques described herein can provide real-time cell viability data, protein and aggregation concentrations and can render the aggregate measurement more accurate. Providing valuable information about structures of interest, protein aggregation, for instance, embodiments described herein can be conducted online, e.g., during an ongoing flow process. Measurements can be obtained rapidly, in situ and in real time, using non-destructive approaches, without the need to withdraw aliquots or divert a portion of the flow.

Some implementations use a device that can be or can include disposable components. Typically, the device can be inserted and/or maintained in a bioreactor or other vessel or transfer line and incorporates elements for viewing as well as elements needed to analyze the contents, e.g., in the NIR region of the electromagnetic spectrum. The analysis can be conducted in real time, in a rapid and nondestructive manner.

In general, according to one aspect, the invention features a system for analyzing for structures in a solution. The system comprises a light source for illuminating the solution, a preferably spatially resolved or image sensor for detecting light from the light source after interaction with the solution, and a controller that analyzes a response of the image sensor to assess the structures in the solution.

In embodiments, the structures are cells and the controller might assess a viability of the cells and/or a number of cells and/or density of the cytoplasm of the cells and/or a density of the cells in their solution.

Other structures can be studied. For example, in some cases, the structures are proteins and the controller might assess aggregation of the proteins, optionally in conjunction with assessing non-aggregated proteins.

The solution can be withdrawn from a vessel for analysis; in other cases, the solution is analyzed while in a bioreactor.

For some implementations, light from the light source is split to additionally perform absorption spectroscopic analysis of the solution.

In some cases, the detector detects light in a transmission arrangement, but in other cases, the detector detects light in a transflection arrangement.

In general, according to another aspect, the invention features a method for analyzing structures in a solution. The method comprises illuminating the solution, detecting light after interaction of the illuminating light with the solution, e.g., using an image sensor, and analyzing a response of the image and possible the speckle to assess the structures in the solution.

The structures or cells can be assessed for their density and/or viability.

Preferably the illuminating light has some coherence. In many implementations, its spatial coherence length should be at least 5 micrometers and its temporal coherence length should be at least 1 millimeter. Many embodiments, employ a light source that is a laser.

In general, according to another aspect, the invention features a probe system for analyzing cells (or other structures) in a bioreactor. This probe system comprises a probe comprising a tip section having a sample detection region, a light source for illuminating the cells in the sample detection region, a sensor for detecting light from the light source after interaction with the cells, and a controller that analyses a response of the sensor to assess cells.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1A is a schematic diagram of a system for laser speckle imaging and analysis for live cell quantification and assessment according to the present invention;

FIGS. 1B, 1C, and 1D are schematic plots showing the intensity of the beam across one axis of the image sensor 126;

FIG. 2 is a schematic diagram showing the offset from the beam center;

FIG. 3 shows several images detected by the image sensor 126 of the camera 124;

FIGS. 4A and 4B are, respectively, plots of mean fluctuation as a function of total cell density (TCD) for a complete image and a subsection of the image for three different pathlengths 0.1, 0.5, and 1 millimeters (mm);

FIG. 5A shows an image detected by the image sensor 126 with an offset beam;

FIG. 5B is a plot of intensity as a function of pixel position (pixel 0 to pixel 1920) along the line scan in FIG. 5A (with background subtracted);

FIG. 6 is a plot of intensity as a function of pixel position (pixel 0 to pixel 1920);

FIG. 7A is a plot of intensity as a function of pixel position (pixel 0 to pixel 1920);

FIG. 7B is a series of plots of intensity as a function of pixel position (pixel 0 to pixel 1920);

FIG. 7C plots the value at the threshold across images, densities and path lengths;

FIG. 8 is a picture of a system for laser speckle imaging for live cell quantification employing a transflection arrangement;

FIG. 9 is a schematic view of an in-situ probe 10 configured for placing and/or maintaining the tip section 14 of the probe 10 within a bioreactor;

FIG. 10 is a processing diagram showing how the controller 200 builds a machine learning model; and

FIG. 11 is a processing diagram showing how the controller 200 employs the machine learning models for determining cell density and viability from the collected speckle images.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In some of its aspects, the invention relates to measuring structures such as cells, including, e.g., their densities, such as the density of their cytoplasm and/or the density of the cells and/or viability within a medium, typically a liquid-containing cell culture. In many implementations, the cells are in an aqueous medium.

Any type of cells can be detected or monitored. Examples include but are not limited to mammalian, bacterial, fungal, and many others.

The life cycle of cells is generally marked by various changes. For instance, before autolysis (i.e., the destruction of cells or tissues by their own enzymes, such as enzymes released by lysosomes) cells will typically stain a deep red. As autolysis progresses, the staining becomes gradually fainter, probably due to losses in stainable material, and the cells appear disorganized. In addition, cells undergoing autolysis have an index of refraction that approaches the index of refraction of the aqueous cell culture, possible due to a loss in cell density (the lowering of the density of the cells' cytoplasm) and/or other mechanisms. In contrast, live cells have an index of refraction that is different from that of the aqueous culture medium, resulting in a turbid environment.

In many of its aspects, the invention relies on laser speckle imaging, a technique based on the random interference pattern produced when coherent light scatters from a medium that can be imaged onto a detector such as a camera. Because of their properties (index of refraction, size, etc., for example) viable cells can give rise or influence interference patterns differently than dead cells (which have a refractive index approaching that of the medium).

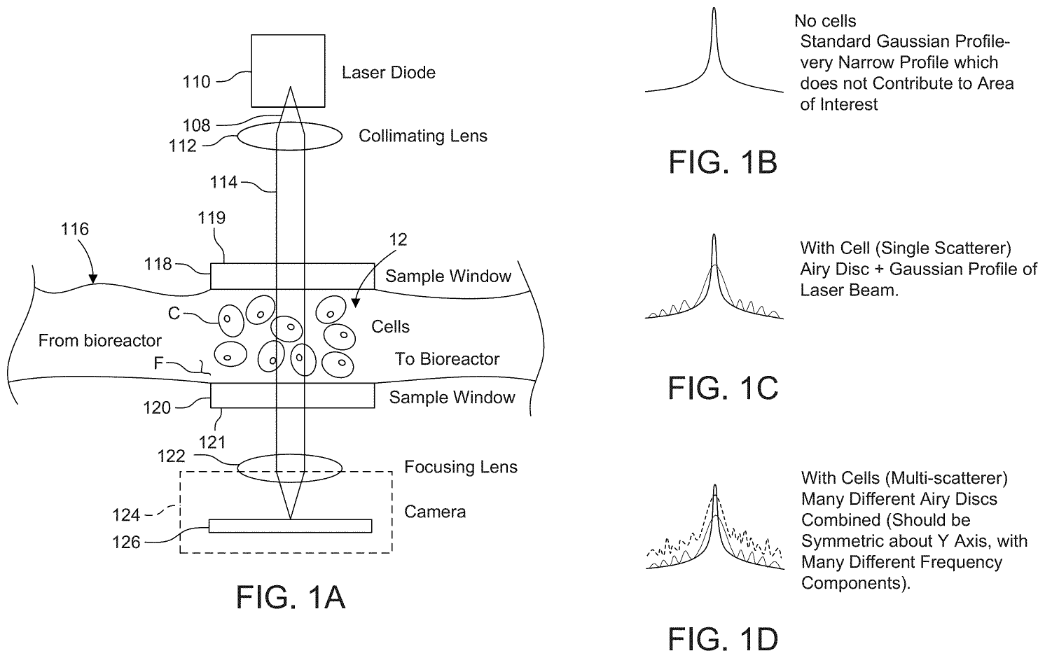

FIG. 1A is a schematic diagram of a system for laser speckle imaging for live cell quantification according to the present invention.

In more detail, an expanding or diverging beam 108 from a laser diode 110, or another coherent light source, is generated. The beam is collimated, if required, by a source lens 112 to form a collimated and coherent light beam 114. The beam 114 enters a flow cell 116 through a first sample window 118 and interacts with the cells C and the fluid F surrounding the cells in a volumetric sample detection region 12 between the first sample window 118 and a second window 120. The light then exits via the second sample window.

A focusing lens 122 focuses the light onto a spatially resolved image sensor 126 of a camera 124. Generally, the image sensor has a resolution of at least 1000 by 1000 pixels.

In many embodiments, the light source is required to have at least some spatial coherence. At a minimum, the spatial coherence length should exceed the lateral dimensions of the structures to be analyzed. In illustrative examples, the spatial coherence length is at least greater than 0.1 micrometers (microns; μm) for the smallest cells. More typically, the spatial coherence length is greater than 5 micrometers or preferably greater such as 10 micrometers, or even greater than 100 micrometers. In some implementations, the spatial coherence length is measured in millimeters and can be greater than 1 millimeter or greater than 10 millimeters.

The light generated by light source 110 also can be characterized by its temporal coherence. In terms of this latter property, it is important that the speckle pattern be detectable with respect to the frame rate of the camera 124. As a result, the temporal coherence length could be minimally greater than 5 micrometers, or greater than 10 micrometers, or even greater than 100 micrometers. In some cases, however, the temporal coherence length is measured in millimeters and can be greater than 1 millimeter or greater than 10 millimeters.

It is possible to coat one or more of the optical components with an antireflective (AR) coating, allowing light to efficiently pass through. In the current embodiment, the exterior surfaces 119 and 121 of the first sample window 118 and the second sample window 120, respectively, are provided with an anti-reflective (AR) coating. This allows light to pass through and focus without some of it being reflected. Examples of suitable AR coatings include but are not limited to thin film dielectric coatings.

FIGS. 1B, 1C, and 1D show the intensity of the beam across one axis of the image sensor 126. This intensity profile is typically the same and symmetric across the other axis of the image sensor.

Specifically, as shown in FIG. 1B, when no cells are present, the intensity profile has a standard Gaussian shape. In contrast, when some cells are present, the intensity profile is affected or modulated by the Airy disc (the bright central spot in the system of diffraction rings formed by an optical system with light from a point source) formed by the lenses, as shown in FIGS. 1C and 1D. Specifically, FIG. 1C illustrates a situation with cell (single scatterer) of an Airy disc superimposed onto the Gaussian profile of the laser beam (the latter as shown in FIG. 1B). FIG. 1D reflects a situation with cells (multi-scatterer) with many Airy discs combined in a symmetrical display about the y-axis, with many different frequency components. When speckle imaging frequency information is obtained in this arrangement, the lens performs a physical Fourier Transform, resulting in information that has size/distribution in space (across the camera) instead of temporal frequency information.

This is caused by different size structures (cells) that will scatter the light at different angles. Specifically, smaller particles will scatter light at larger angles and larger particles will scatter light closer to the main (incident) beam (at smaller angles).

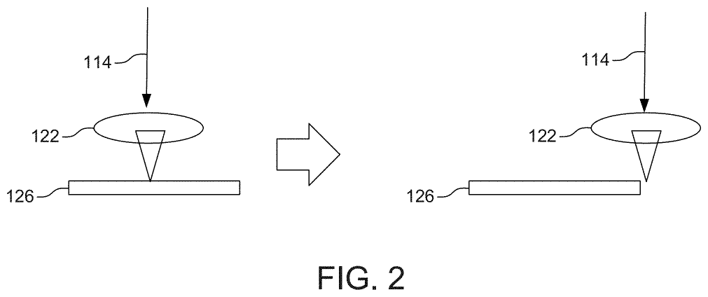

FIG. 2 shows a preferred implementation detail. Since the camera is seeing frequency across its image sensor 126, the center of the beam 114 is essentially the “DC” component of a Fast Fourier Transform (FFT). To avoid measuring this and thus burning out the camera, the beam center is moved just off the camera.

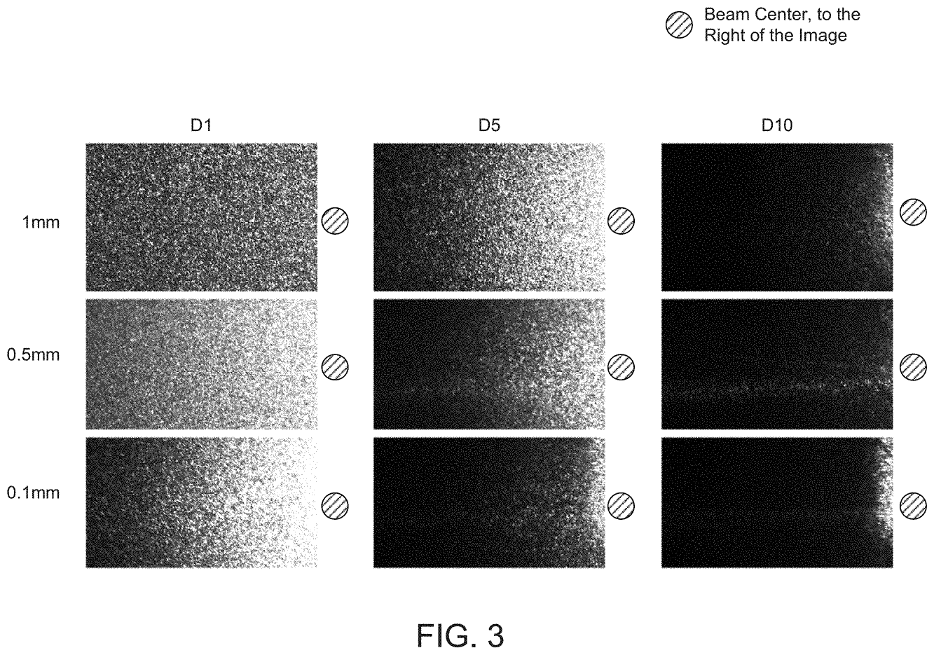

FIG. 3 shows a matrix of several images detected by the image sensor 126 of the camera 124.

In these images from the volumetric sample detection region 12, the cells were diluted from 33×10⁶/mL (D1) to 0.06×10⁶/mL (D10). The dilutions were 1:1, made sequentially for 10 scans (D1 being the most concentrated and D10 the least concentrate). The data cover different pathlengths, 1 millimeter (mm), 0.5 mm, and 0.1 mm to show differences in dynamic range. The beam center is to the right of each image.

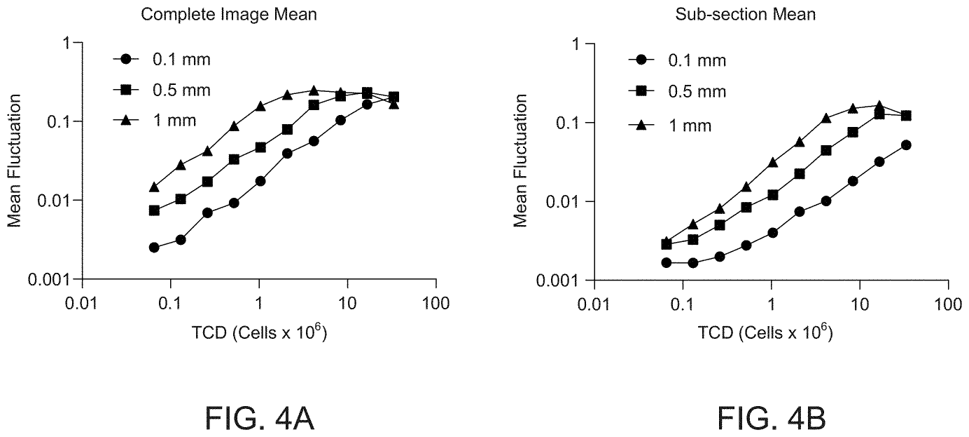

FIGS. 4A and 4B are plots of mean fluctuation as a function of total cell density (TCD) for a complete image and a subsection of the image for three different pathlengths 0.1, 0.5, and 1 mm. In general, the intensity fluctuates both spatially and temporally. The plots show the mean of that fluctuation across (left) the entire image (FIG. 4A) and (right) a subsection of the image (FIG. 4B). The data indicates that the mean fluctuation is correlating to the amount of structures present, expressed in this case as total cell densities (TCD).

Utilizing the fluctuations (since what is captured is similar to movies), the mean is calculated by subtracting the background (just cell culture medium) and then examining the standard deviation in time, while taking the average across either the entire image (complete image mean) or a sub-section such as a part of the middle of the image.

Choosing where the mean is selected can change the dynamic range (where the signals are linear with cell density).

As expected, a longer path length has more linearity at lower cell densities, and a smaller path length has more linearity at higher cell densities.

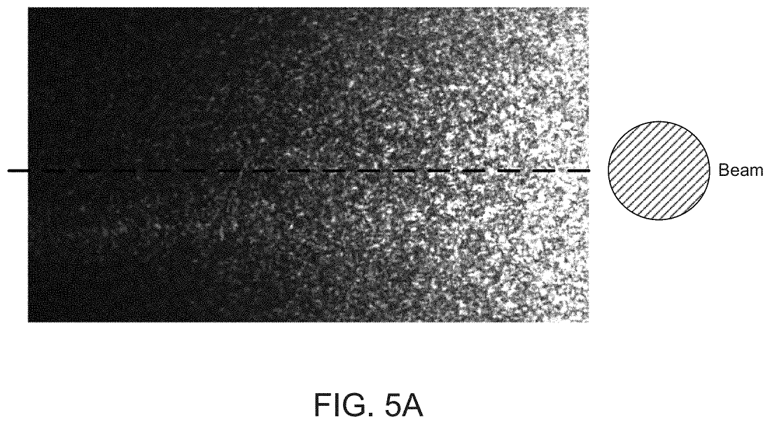

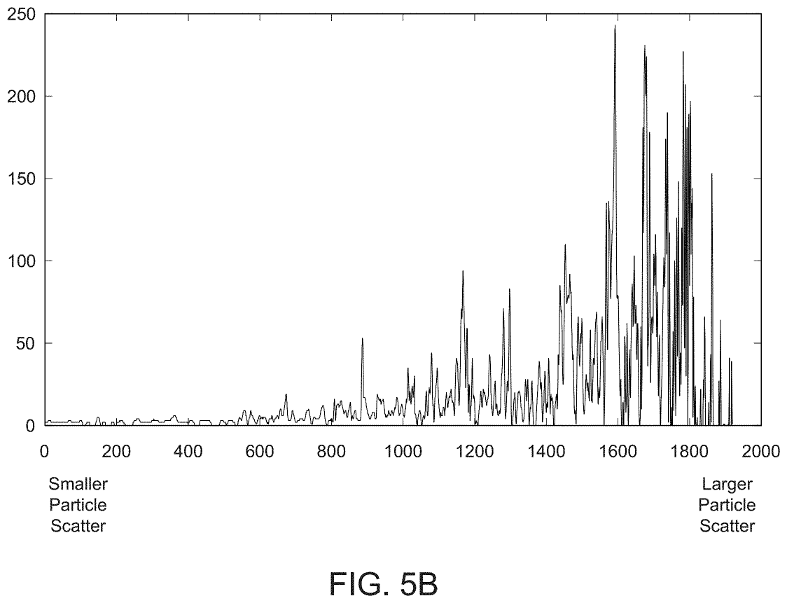

FIG. 5A shows an image detected by the image sensor 126 with the off-center beam from the laser; FIG. 5B is a plot along the line scan (with background subtracted). This is the summation of Airy discs with background subtracted. In some cases, it may be possible to subtract mean of a time series to fluctuate around 0.

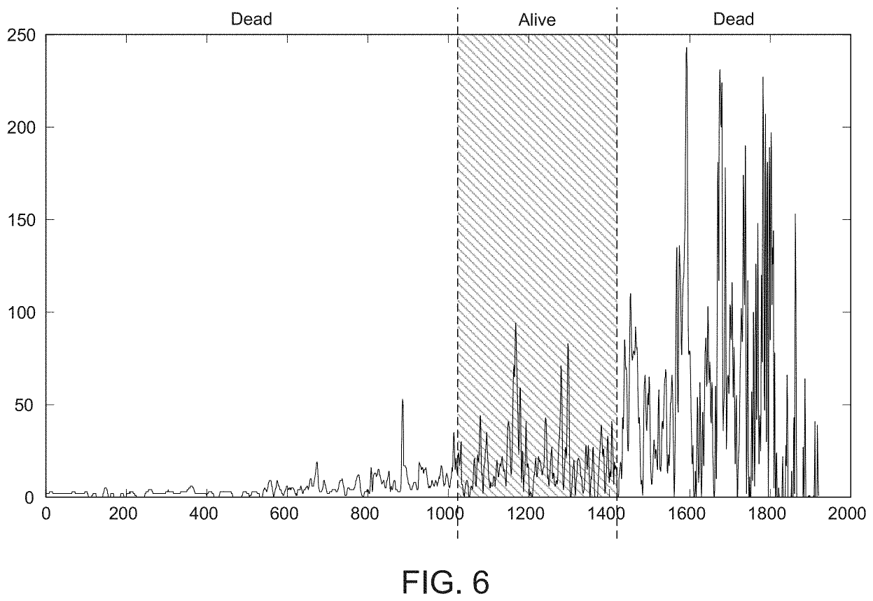

FIG. 6 shows a plot (intensity versus pixel position) along the line scan (with background subtracted) showing the “alive” and “dead” areas.

An initial calibration can be provided for unknown sizes to create a cutoff (or window) for what can be thought as “healthy” cells. The calibration discounts cells that are really small (they would be dead) or really big, as they would also be dead. A spatial thresholding is used to determine where and how live/dead cells scatter. Then, a model can be built upon this analysis to estimate future batches.

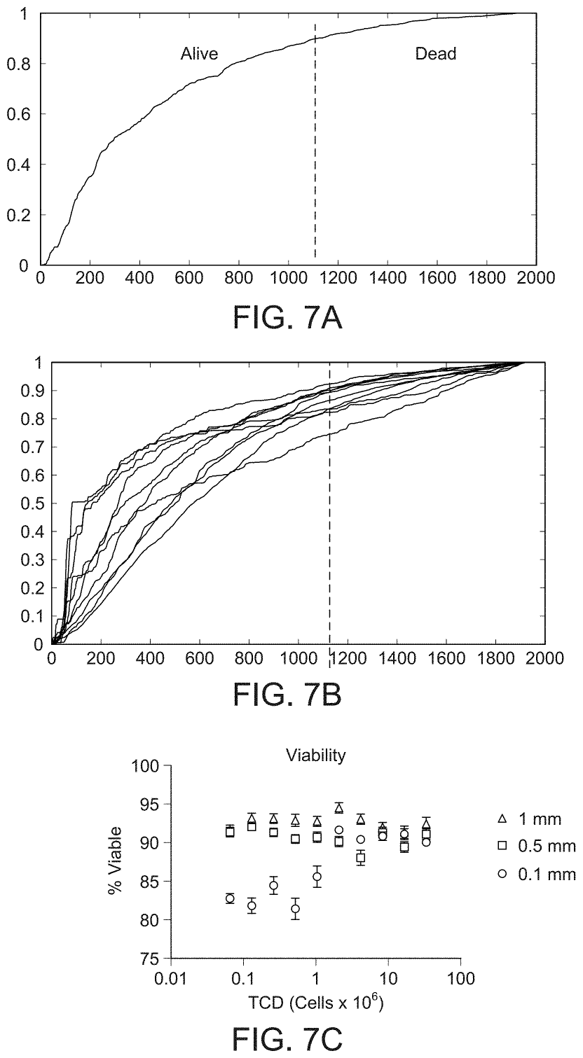

FIG. 7A shows a plot of the cumulative integral (y axis) of FIG. 6.

FIG. 7B shows different plots (y-axis represents the integral, x-axis the pixels) corresponding to different cell densities. The samples were prepared by adding cells at different densities.

It shows the cumulative integral (in pixel space) divided by the total integral to normalize image to image and remove different intensity from scatter. Showing this across all densities yields the separate plots.

FIG. 7C plots the value at the threshold across images, densities and path lengths. In more detail, the % viable cells as a function of TCD values are shown for three pathlengths, namely 1 mm, 0.5 mm and 0.1 mm.

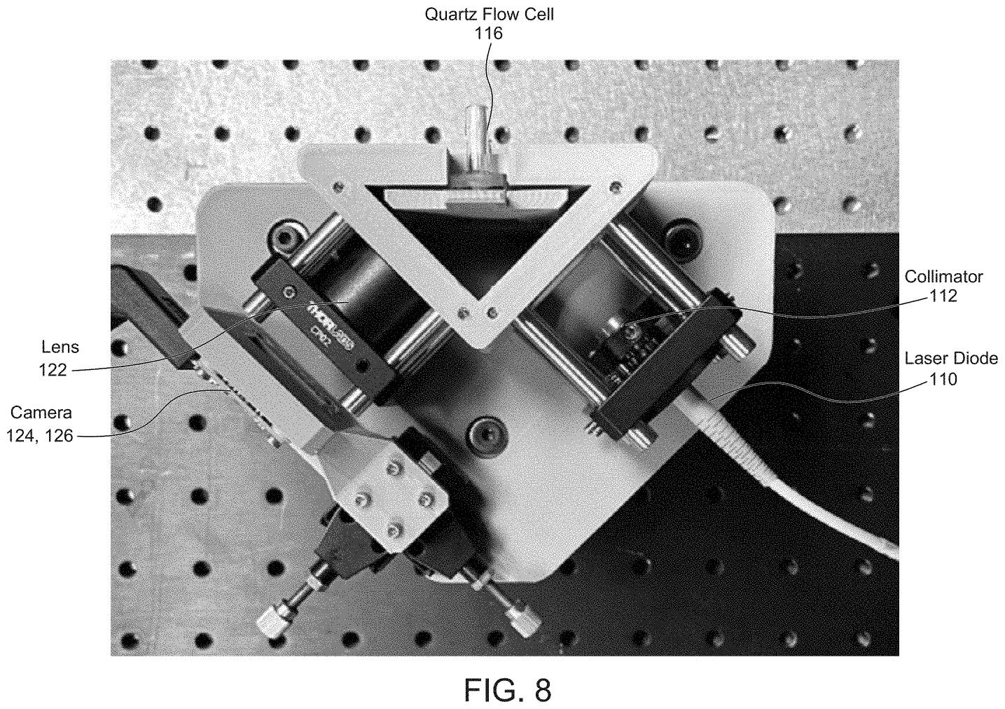

FIG. 8 is a picture of a system for laser speckle imaging for live cell quantification employing a transflection arrangement. As seen in this figure, the beam is generated by the laser diode or another suitable light source (e.g., another type of laser) 110. If necessary, the beam is collimated by the source collimation lens 112 to form a collimated light beam. The beam enters the flow cell 116, made of quartz, for example, and is reflected by a mirror, such as gold coated substrate, or by the contents of the flow cell. The focusing lens 122 focuses the light onto a spatially resolved image sensor 126 of a camera 124.

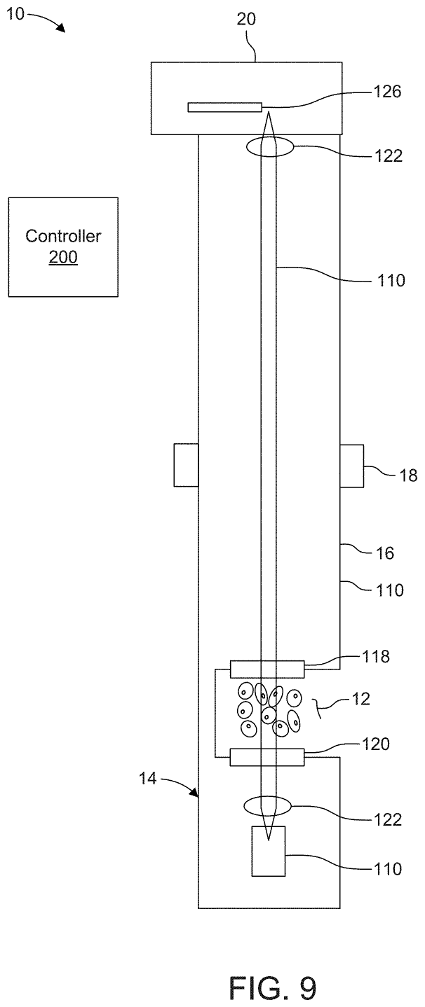

FIG. 9 shows an in-situ probe 10 in a schematic cross-section. Probe 10 is configured for placing and/or maintaining the tip section 14 of the probe within the bioreactor. The tip section can be made of stainless steel and includes a sample detection area or region 12 which can be shaped with an indentation, as shown in FIG. 9.

Since many bioreactor headplates are equipped with ports for receiving various fittings which can be screwed in, a fitting 18 including an optional Teflon washer and EPDM rubber O-ring, or another suitable arrangement, can be used to provide a seal on the headplate at the top of a bioreactor. In more detail, the fitting can be a PG13.5 18 fitting having the standard thread typically used on bioreactor headplates. Optional Teflon washer (which does not provide a seal) can be used as a spacer to ensure sealing on certain bioreactor headplates that may have a deeper threaded section for the PG13.5 fitting. The EPDM O-ring creates the seal between the headplate and the bottom of the Teflon washer as the PG13.5 fitting 18 is tightened.

The dimensions of tip section 14 and an outer tube 16 can be selected according to the size of the reactor. In many situations, the longitudinal distance between the fitting 18 and the volumetric sample detection region 12 between the first sample window 118 and a second window 120 of the tip section 14 is configured to expose sample detection region 12 to the reactor medium being monitored and specifically a portion of that medium that is representative of all of the medium in the bioreactor, rather than possibly unmixed medium along a wall of the reactor. In one illustrative example, the distance between the fitting 18 and the optical detection region 12 is at least 1 centimeter (cm), e.g., at least 2, 3, 4, 5, 10, 15, 20, 25, 30, 35, 40, 45, 50, 55, 60, 65, 70, 75, 80, 85, 90, 95 or 100 cm. Tip section 14 can be smaller, for miniaturized reactor designs, for instance, or larger, for some industrial scale applications.

For analysis, a light beam (generated by a source laser 110) is collimated by lens 112. The light propagates through the bioreactor medium present in a volumetric sample detection region 12 and reaches the image sensor 126 in a head section 20. Surfaces of windows 118 and 120 that contact the reactor medium define the pathlength travelled by the coherent laser light and the indentation forming the sample region 12 can be dimensioned accordingly.

A controller 200 monitors the response of the image sensor 126. The detachable electrical connections are made through a multi-pin connector. Thus, the controller 200 can resolve the response of the sample, specifically the interaction between the coherent light from the laser 110 and structures in the sample detection region 12, and the resulting speckle patterns formed on the image sensor 126.

According to the invention, the controller 200 resolves spatial and temporal contrast and these contrast changes are then used to determine the presence of viable cells, the density of the cells, and the viability of those cells.

The sampling and analysis by the controller 200 can be repeated with any desired frequency over any desired time period. For example, viable cells numbers can be measured daily or at intervals of 2, 3 or more days for a week, two weeks, three weeks or longer. The presence of other constituents can be determined every few minutes, e.g., every 5 minutes. Thus, the entire process can be monitored in real time.

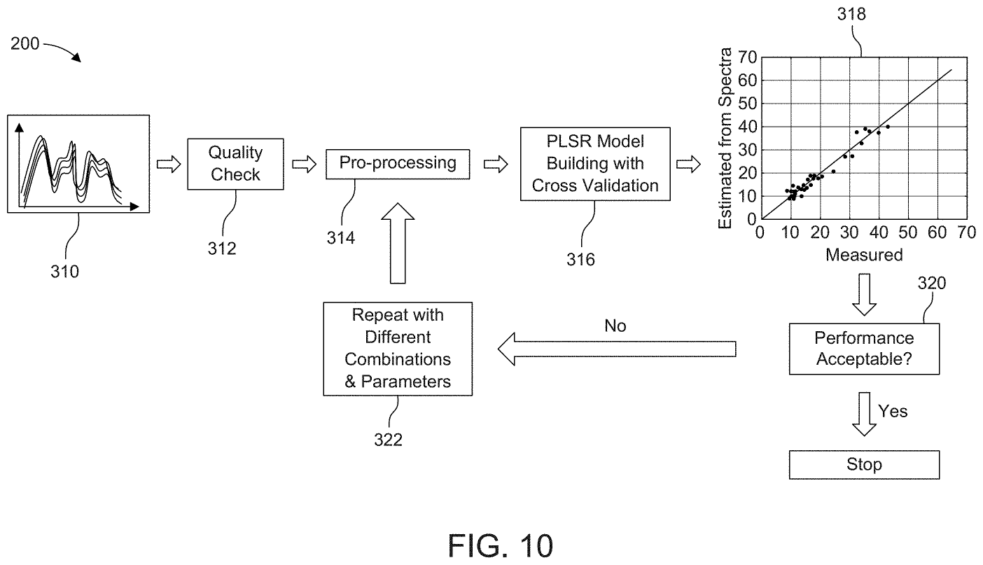

In some embodiments, the controller 200 executes a machine learning system or protocol to assess the state of the cells. This is illustrated in FIG. 10, which is a processing diagram showing how the controller 200 builds a machine learning model for determining cell density and viability from the collected speckle images.

In more detail, the images 310 are first analyzed in a quality check operation 312. This ensures that the images are generally consistent with what was expected. Then, in a preprocessing step 314, the images are often normalized with respect to each other. Some spatial bandpass filtering also can be conducted at this stage.

A partial least squares regression model can be built, in step 316. Cross validation is preferably employed. Validation is determined in step 318 by considering how well the model predicts the density of cells and assesses the viability of those cells against the measurements for those parameters.

Step 320 determines if the performance is acceptable. If a model's performance is unacceptable, then in step 322, the model building is repeated with different combinations and parameters.

In some cases, models obtained as described above or by other techniques can be tested by comparing their results with data collected using conventional analytical techniques, utilizing fluorescent dyes, for instance.

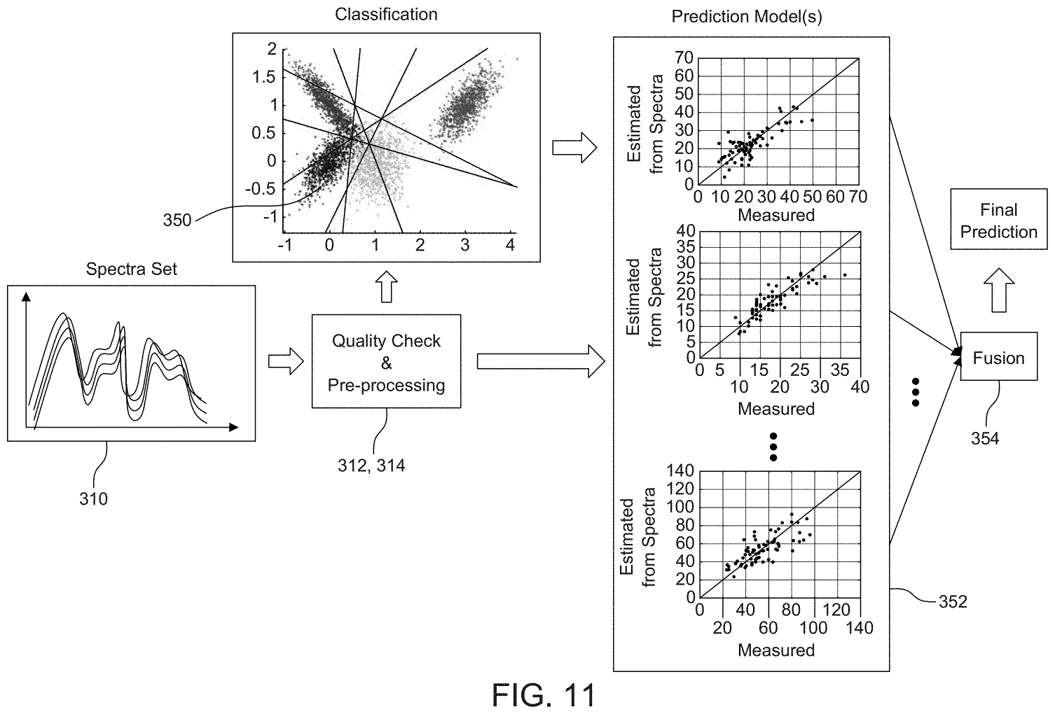

FIG. 11 is a processing diagram showing how the controller 200 employs the machine learning models for determining cell density and viability from the collected speckle images.

In more detail, the images 310 are also analyzed in a quality check operation and preprocessed in steps 312, 314, described above. Then the classification is applied in one or more models generated as described in connection with FIG. 10.

The results of the one or more prediction models 352 are then fused in a fusion step 354 to produce the final result.

Viable cells can be analyzed in other situations, for example, in the detection of bioburden in wastewater treatment, in protein production in the laboratory or the commercial manufacture of proteins, and so forth.

Techniques such as described herein also have the potential to enter the cell and gene therapy production process and provide important insight into cell quality and therapy development.

In other aspects, the invention can provide information concerning structures other than cells, for example proteins, protein aggregates and so forth. In one embodiment, the invention relates to assessing, within a fluid, the level of aggregation encountered during the preparation of proteins, such as, for instance, monoclonal antibodies, IgG, IgA, IgM, TNF-alpha, IFN-gamma, to list a few.

Specific embodiments relate to detecting and, preferably, quantifying, protein aggregates. As used herein, the term “protein aggregates” refers to oligomers such as protein dimers, trimers, higher oligomers, polymers, clusters, filaments, agglomerated aggregates and/or other assemblies formed from the monomeric form of the protein. A study of various possible aggregated states that can be adopted by proteins, either as folded molecule or unfolded/partially-unfolded ones, is provided by C. J. Roberts in Protein Aggregation and Its Impact on Product Quality, Curr. Opin. Biotechnol., 2014 December; 0: 211-217, published online 2014 Aug. 28, PMC4266928, which is incorporated herein by this reference. Factors identified in this publication as contributing to whether or how rapidly aggregation can occur include solution conditions (pH, salt concentration and/or the nature of salt employed, the amount and type of osmolytes present, the use of amphiphilic molecules such as surfactants); temperature; pressure; air-water interfaces; other bulk interfaces (e.g., between water and stainless steel), and so forth.

For example, when heated, some proteins will undergo aggregation, first forming soluble assemblies (such as dimers, trimers, other small oligomers), which can then progress, often rapidly, to insoluble large or very large fibrils. Since, in practical terms, it might be possible to control the formation of insoluble protein assemblies (by adjusting solution conditions, for example), some embodiments described herein focus on detecting soluble protein aggregates. Techniques described herein may also be employed to detect larger, insoluble species, e.g., contaminants.

Monitoring protein aggregates can be conducted in conjunction with detecting and, preferably, quantifying amounts of the protein in its monomeric, also referred to herein as “unaggregated”, form. Measuring both unaggregated protein and protein aggregates can be simultaneous or nearly simultaneous.

In one illustrative example, the invention is applied to the production of human or humanized IgG monoclonal antibodies (e.g., IgG1, IgG2 or IgG4). An initial protein product (obtained, for instance, by separating cells from their culture medium) is purified and concentrated in a single “capture” operation based on protein A chromatography. While Staphylococcal protein A can provide very efficient binding to IgG molecules, elution of the product typically relies on lowering the pH. Since acidic conditions and abrupt changes in pH have been associated with aggregation (see, e.g., A. R. Mazzer et al., Protein A Chromatography Increases Monoclonal Antibody Aggregation Rate During Subsequent low pH Virus Inactivation Hold, J. Chromatogr. A, 1415:83-90, Oct. 9, 2015), assessing the degree of aggregation at this point in the production process can be important.

Thus, techniques described herein can be particularly useful when conducted on fluid eluted from protein A columns. Other applications pertain to streams that are cleaner and/or purer, such as those that can be produced by cation exchange columns (CEX), for example. Another use is ultrafiltration (UF) and diafiltration (DF), which are steps in downstream processing for product concentration and buffer exchange. In an exemplary process, UF/DF precede chromatographic columns with this UF/DF pre-treatment preparing the product for the subsequent chromatography stage. The columns might be operating under different pH or molarity conditions. Typically, the UF/DF steps concentrate and resuspend the product in the correct buffer before introduction into the columns.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for analyzing structures in a solution, the system comprising:
a light source for illuminating the solution;
a sensor for detecting light from the light source after interaction with the solution; and
a controller that analyses a response of the sensor to resolve speckle information to assess the structures in the solution.

2. The system as claimed in claim 1, wherein the structures are cells.

3. The system as claimed in claim 2, wherein the cells are in or from a bioreactor.

4. The system as claimed in claim 2, wherein the controller assesses a viability of the cells.

5. The system as claimed in claim 1, wherein the controller assesses a density of the cells.

6. The system as claimed in claim 1, wherein the light source is a laser.

7. The system as claimed in claim 1, wherein the light source has spatial coherence length of at least 5 micrometers.

8. The system as claimed in claim 1, wherein the light source has temporal coherence length of at least 1 millimeter.

9. The system as claimed in claim 1, wherein the sensor is an image sensor having a resolution of at least 1000 by 1000 pixels.

10. A probe system for analyzing cells in a bioreactor, the probe system comprising:
a probe comprising a tip section having a sample detection region;
a light source for illuminating cells in the sample detection region;
a sensor for detecting light from the light source after interaction with the cells in the sample detection region; and
a controller that analyses a response of the sensor to assess the cells in the sample detection region.

11. The probe as claimed in claim 10, wherein the sensor is an image sensor having a resolution of at least 1000 by 1000 pixels.

12. The probe as claimed in claim 10, wherein the light source is a laser.

13. The probe as claimed in claim 10, wherein the light source has spatial coherence length of at least 5 micrometers.

14. The probe as claimed in claim 1, wherein the light source has temporal coherence length of at least 1 millimeter.

15. A method for analyzing structures in a solution, the method comprising:
illuminating the solution;
detecting light after interaction with the solution; and
analyzing speckle information to assess the structures in the solution.

16. The method as claimed in claim 15, wherein light illuminating the solution has spatial coherence length of at least 5 micrometers.

17. The method as claimed in claim 15, wherein light illuminating the solution has temporal coherence length of at least 1 millimeter.

18. The method as claimed in claim 15, wherein the structures are cells, proteins or protein aggregates.

19. A method for analyzing for cells in a bioreactor, the method comprising:
illuminating the cells in a sample detection region in the bioreactor;
detecting light from the light source after interaction with the cells; and
analyzing a response of the sensor to assess the cells.

20. The method as claimed in claim 19, wherein the response of the sensor provides speckle information.

21. The method as claimed in claim 20, further comprising correlating the response of the sensor to total cell densities and/or viable cell percentages.
